(19) United States
(12) Patent Application Publication
(10) Pub. No.: US 2019/0038198 A1
(43) Pub. Date: Feb. 7, 2019

(54) MEDICAL DEVICE

(71) Applicant: Keri Lee Ingram, Fort White, FL (US)

(72) Inventor: Keri Lee Ingram, Fort White, FL (US)

(21) Appl. No.: 15/665,498

(22) Filed: Aug. 1, 2017

(51) Int. Cl. A61B 5/15; A61M 5/315

(52) U.S. Cl. CPC A61B 5/150473; A61B 5/150244; A61M 21/02; A61M 5/31511; A61B 5/150732; A61B 5/150389

(57) ABSTRACT

A medical device having an attachment means and a stimulating probe. The stimulating probe is separated from the attachment means by a length of flexible tubing. The flexible tubing has a hollow interior configured to direct the flow of water.

### CROSS-REFERENCE TO RELATED APPLICATION(S)

Not applicable

### BACKGROUND OF THE INVENTION

### 1. Field of Invention

The present invention relates to the field of medical devices, and more particularly to medical devices used for stimulation and stress relief.

### 2. Description of Related Art

Comprehensive healthcare relies on preventative and conscious efforts of the individuals to promote health practices. Specific elements of a person's wellbeing would include physical, mental, and emotional health. Physical health would include a proper diet and exercise. Physical health is integrated into metal and emotional health through the release of hormones and regulatory biochemical compounds within the body. For example, the release and regulation of serotonin, epinephrine and norepinephrine can be performed through certain physical activity.

Sexual stimulation is a particularly useful physical activity considering the hormone activity and intimate nature of the acts involved. Biological action potentials involve controlled release of hormones and musculature engagement.

The intimacy involved in sexual related activities add to the biological impact the activity has on the individual. Further, the intimacy can simulated through the use of prosthetic devices configured to replicate body parts or objects configured to target organs related to the sexual action potential. The action potential relates to the amount of stimulation required to overcome such that the individual would orgasm.

Currently, passive devices are provided requiring consistent amounts of user control. Automated devices attempting to alleviate the required user control are limited in their capability to dynamically stimulate the user. Further, the synthetic mechanisms are generally limited based on the portability or battery operation of the device.

Based on the foregoing, there is a need in the art for a device that will provide for realistic replication of biologically stimulating components. A device is needed with a multifunctional capability of providing dynamic stimulation, resulting in reduced stress and improved health of the user.

### SUMMARY OF THE INVENTION

A medical device having an attachment means and a stimulating probe. The stimulating probe is separated from the attachment means by a length of flexible tubing. The flexible tubing has a hollow interior configured to direct the flow of water.

In an embodiment, the attachment means comprises one or more valves to selectively control the direction of water flowing from a water source.

In an embodiment, the stimulating probe further comprises a stimulation element within an interior of the stimulating probe.

In an embodiment, the stimulation element is hydraulically operated, wherein water having pressure enters the stimulation probe and the pressure drives the stimulation element.

In an embodiment, the stimulation element comprises a plurality of bearings configured to rotate within the interior of the stimulating probe, wherein the stimulating probe is resilient, and wherein kinetic energy of the plurality of bearings is transferred through to an exterior surface of the stimulating probe.

In an embodiment, the stimulation element comprises a plurality of tubes extending into an interior of the stimulating probe, wherein a proximal end of the plurality of tubes is in communication with water from a water source, wherein a distal end of the plurality of tubes is open to the interior of the stimulating probe.

In an embodiment, the stimulating probe is comprised of a thermally reactive material, wherein the stimulating probe changes color based on a temperature of water entering an interior of the stimulating probe.

In an embodiment, the stimulating probe is configured to emulate a penis.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the medical device, according to an embodiment of the present invention;

FIG. 2 is a perspective view of the medical device, according to an embodiment of the present invention;

FIG. 3 is a perspective view of the medical device, according to an embodiment of the present invention; and

FIG. 4 is a perspective view of the medical device, according to an embodiment of the present invention.

### DETAILED DESCRIPTION OF PREFERRED EMBODIMENTS

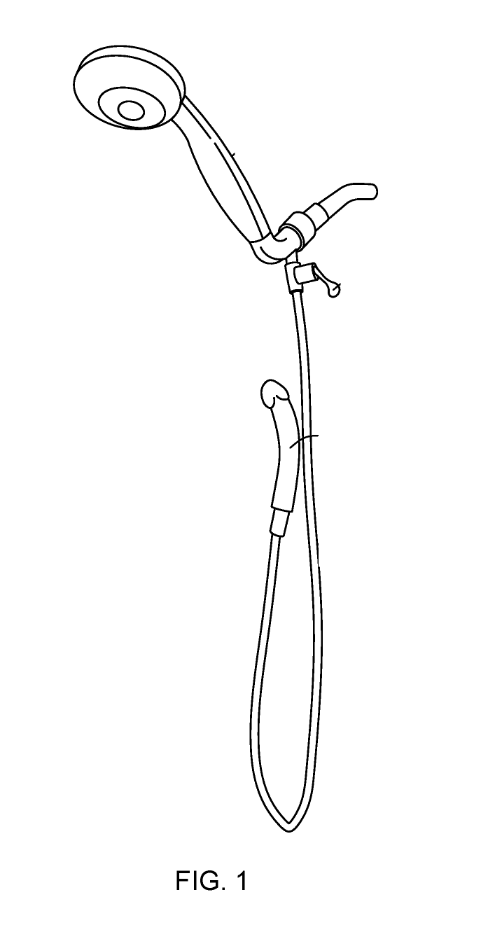

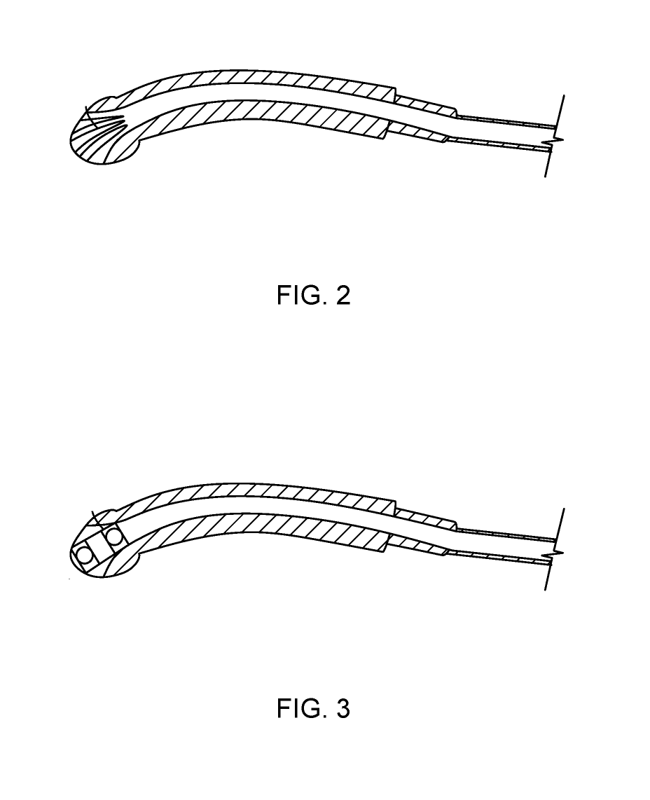

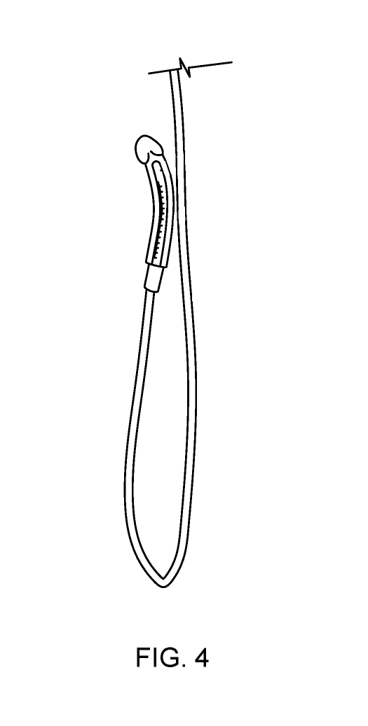

Preferred embodiments of the present invention and their advantages may be understood by referring to FIGS. 1-4.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

A medical device has a probe portion in communication with a water source. The water source provides for hydraulic operation of the probe, where the probe is configured to engage a user. A flexible member extends between a distal end of the probe portion and the water source. The flexible member has a hollow interior and directs the flow of water from the water source to the probe end. The water enters the probe end and hydraulically activates operation of the probe portion. A bifurcated valve is attached to the water course wherein one stem of the bifurcations is attached to the flexible member and the second stem is attached to a shower head or faucet. One or more valves may be disposed on each stem to selectively control the flow of water once the device has been installed.

The probe portion may have an irregularly textured exterior. The proximal end of the probe portion is configured to be inserted into a vaginal canal of the user. In such a configuration, the proximal end of the probe portion may comprise a bulbous section substantially near the proximal end.

The exterior surface of the probe portion, between the bulbous portion and the distal end may be smooth. However, in some embodiment, the exterior surface is non-uniform. For example, the exterior surface may provide for a plurality of ribs defined by raised sections circumferentially engaged to the exterior surface. In an alternative embodiment, simulated expressed veins may extend between the bulbous portion and the distal end.

In an embodiment, the simulated expressed veins are provided when the water source supplies a sufficient amount of water pressure to the probe portion. In such an embodiment, the water pressure forces the sections of simulated expressed veins to protrude outwards from the exterior surface of the probe portion.

In an embodiment, the distal end of the probe portion attaches to the flexible member through a threading engagement or a quick disconnect engagement, either of which are water tight.

In another embodiment, a system of tubing or channels are disposed within the probe portion directing the flow of water within the probe portion. The flow of water may be directed through one or more mechanical mechanisms within with the probe portion.

Mechanical mechanisms within the probe portion are hydraulically operated. The water pressure engages portions of the mechanisms thereby displacing various elements within the probe portion. For example, the mechanism mechanism may provide for a plurality of spherical elements moveably arranged within various segments of the probe portion. The water enters the interior of the probe portion and is directed to contact an initial spherical element. The spherical elements receive kinetic energy from the water and transfers the energy to the remaining spherical elements resulting in their movement within the probe portion. Movement of the spherical elements within the probe portion result in contact with the interior walls of the probe portion and therefore provide momentary undulation or projections through to the exterior resulting in a dynamic and changing exterior surface.

Another example of a mechanical mechanism involves a rotatable element having a central point coaxially aligned with a longitudinal axis of the probe portion. The rotatable element is moved by the flow of water within the probe portion such that the element rotates resulting in vibration of the probe portion transferred from the rotation and vibration of the rotatable element.

Another example of a mechanical mechanism within the probe portion provides for a plurality of tubes separating from a main branch. In such an example, the interior of the probe portion is hollow and the walls of the probe are sufficiently thin to allow for transfer to contact from the tube ends. The plurality of tubes each terminated near the proximal end of the interior of the probe portion. The terminal ends of the tubs are open allowing for water from the main branch to flow out of the ends of the plurality of tubes. The flow of water within the tubes from the main branch to the terminal ends results in articulation and rapid movement of the plurality of tubes. The movement results in contact with the exterior walls of the probe and the contact then is felt as vibration or dynamic configurations of the exterior surface.

In an alternative embodiment, a valve is disposed at or near the distal end of the probe portion. The vale is configured to selectively adjust or control water pressure within the device. The water pressure may buildup in the probe portion and the vale may be opened allowing for the flow of water out from the probe portion.

In yet another embodiment, a tube is disposed within the flexible members and the internal tube system within the probe portion. The tube acts to direct the flow of water from the probe portion back to the water source. Where the water source is a shower head, the tube portion direct the flow of water through the shower head allowing the user to shower while using the probe portion.

In an alternative embodiment, one or more apertures extends between the hollow interior of the probe portion and the exterior surface. The apertures are configured to allow a predetermined volume of water to flow from the interior to outside of the probe portion.

In an alternative embodiment, the probe is comprised of a thermally reactive material. The material reacts to varying temperatures of water disposed within the probe portion. As the temperature changes, the color of the probe portion changes providing for a visual indication of the temperature to the user. This way, the user can add a further stimulus of temperature of the probe to enhance the stimulation in use.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A medical device comprising:
a. an attachment means; and
b. a stimulating probe, wherein the stimulating probe is separated from the attachment means by a length of flexible tubing, wherein the flexible tubing has a hollow interior configured to direct the flow of water.

2. The device of claim 1, wherein the attachment means comprises one or more valves to selectively control the direction of water flowing from a water source.

3. The device of claim 1, wherein the stimulating probe further comprises a stimulation element within an interior of the stimulating probe.

4. The device of claim 3, wherein the stimulation element is hydraulically operated, wherein water, having pressure, enters the stimulation probe and the pressure drives the stimulation element.

5. The device of claim 3, wherein the stimulation element comprises a plurality of bearings configured to rotate within the interior of the stimulating probe, wherein the stimulating probe is resilient, and wherein kinetic energy of the plurality of bearings is transferred through to an exterior surface of the stimulating probe.

6. The device of claim 3, wherein the stimulation element comprises a plurality of tubes extending into an interior of the stimulating probe, wherein a proximal end of the plurality of tubes is in communication with water from a water source, wherein a distal end of the plurality of tubes is open to the interior of the stimulating probe.

7. The device of claim 1, wherein the stimulating probe is comprised of a thermally reactive material, wherein the stimulating probe changes color based on a temperature of water entering an interior of the stimulating probe.

8. The device of claim 1, wherein the stimulating probe is configured to emulate a penis.
